(19) United States
(12) Patent Application Publication
(10) Pub. No.: US 2024/0127933 A1
(43) Pub. Date: Apr. 18, 2024

(54) Immunization Management System and Method

(71) Applicant: MyDigiRecords LLC, Kansas City, MO (US)

(72) Inventors: Saroj Gupta, Overland Park, KS (US); Shekhar Gupta, Overland Park, KS (US)

(21) Appl. No.: 17/967,959

(22) Filed: Oct. 18, 2022

(51) Int. Cl. G16H 40/20; G06F 40/58; G06V 30/19; G10L 15/18; G10L 15/22; G16H 10/60

(52) U.S. Cl. CPC G16H 40/20; G06F 40/58; G06V 30/19; G10L 15/18; G10L 15/22; G16H 10/60; G06Q 50/30

(57) ABSTRACT

A system includes a computing device to receive medical information associated with a particular patient, compare immunization records for the particular patient with a recommended immunization schedule for the particular patient and determine that there is at least one immunization to be scheduled for the particular patient, schedule the at least one immunization for the particular patient based on at least one factor comprising a spoken language of the particular patient, a location of the particular patient, and acceptance of insurance for the particular patient for a healthcare provider, and generate an alert in realtime for the healthcare provider when the particular patient arrives at the healthcare provider for the at least one immunization scheduled for the particular patient.

### BACKGROUND

For many people, it is especially difficult to stay up to date with recommended vaccines. In many instances, there can be a variety of issues that can prevent people from being able to receive vaccines that can eliminate or substantially reduce the risks of various diseases and illnesses. In one example, a patient may have financial issues that prevent them from being able to receive vaccines. In another example, a patient may not be insured or may be under-insured. As another example, a patient may be very young, elderly, or not have the ability to travel to receive immunizations. In other examples, a patient, caregiver, or family member may not speak a native language of a particular country, may have hearing, vision, or mobility difficulties, or may be unable to communicate with healthcare providers for other reasons.

It is with these issues in mind, among others, that various aspects of the disclosure were conceived.

### SUMMARY

The present disclosure is directed to an immunization management system and method. A system may include a client computing device that communicates with one or more server computing devices to manage immunization and medical records for a user or patient. The system may provide automatic management of patient records and increase a patient's quality of life by making it vastly easier to receive vaccines that are recommended and scheduled for a particular patient. The system may receive patient information for a particular patient that may include immunization information and medicine information, among other information. The system may determine one or more recommended vaccinations for the particular patient and may schedule the one or more recommended vaccinations for the particular patient.

In one example, a system may include a computing device comprising a memory storing computer-readable instructions and at least one processor to execute the instructions to receive medical information associated with a particular patient, the medical information comprising immunization records and current medicines prescribed for the particular patient, compare the immunization records for the particular patient with a recommended immunization schedule for the particular patient and determine that there is at least one immunization to be scheduled for the particular patient, schedule the at least one immunization for the particular patient based on at least one factor comprising a spoken language of the particular patient, a location of the particular patient, and acceptance of insurance for the particular patient with a healthcare provider, and generate an alert in realtime for the healthcare provider when the particular patient arrives at the healthcare provider for the at least one immunization scheduled for the particular patient by determining a location of the computing device and sending the alert to the healthcare provider, the alert indicating at least one conflict for the at least one immunization based on the medical information.

In another example, a method may include receiving, by at least one processor of a mobile computing device, medical information associated with a particular patient, the medical information comprising immunization records and current medicines prescribed for the particular patient, comparing, by the at least one processor of the mobile computing device, the immunization records for the particular patient with a recommended immunization schedule for the particular patient and determining that there is at least one immunization to be scheduled for the particular patient, scheduling, by the at least one processor of the mobile computing device, the at least one immunization for the particular patient based on at least one factor comprising a spoken language of the particular patient, a location of the particular patient, and acceptance of insurance for the particular patient with a healthcare provider, and generating, by the at least one processor of the mobile computing device, an alert in realtime for the healthcare provider when the particular patient arrives at the healthcare provider for the at least one immunization scheduled for the particular patient by determining a location of the computing device and sending the alert to the healthcare provider, the alert indicating at least one conflict for the at least one immunization based on the medical information.

In another example, a non-transitory computer-readable storage medium includes instructions stored thereon that, when executed by a computing device cause the computing device to perform operations, the operations including receiving medical information associated with a particular patient, the medical information comprising immunization records and current medicines prescribed for the particular patient, comparing the immunization records for the particular patient with a recommended immunization schedule for the particular patient and determining that there is at least one immunization to be scheduled for the particular patient, scheduling the at least one immunization for the particular patient based on at least one factor comprising a spoken language of the particular patient, a location of the particular patient, and acceptance of insurance for the particular patient with a healthcare provider, and generating an alert in realtime for the healthcare provider when the particular patient arrives at the healthcare provider for the at least one immunization scheduled for the particular patient by determining a location of the computing device and sending the alert to the healthcare provider, the alert indicating at least one conflict for the at least one immunization based on the medical information.

These and other aspects, features, and benefits of the present disclosure will become apparent from the following detailed written description of the preferred embodiments and aspects taken in conjunction with the following drawings, although variations and modifications thereto may be effected without departing from the spirit and scope of the novel concepts of the disclosure.

### BRIEF DESCRIPTION OF THE DRAWINGS

The accompanying drawings illustrate embodiments and/or aspects of the disclosure and, together with the written description, serve to explain the principles of the disclosure. Wherever possible, the same reference numbers are used throughout the drawings to refer to the same or like elements of an embodiment, and wherein:

FIG. 1 is a block diagram of an immunization management system according to an example of the instant disclosure.

FIG. 2 is a block diagram of a client computing device having an immunization management application according to an example of the instant disclosure.

FIG. 3 is a flowchart of a method of scheduling at least one immunization for a particular patient according to an example of the instant disclosure.

FIGS. 4-8 show example screenshots of a graphical user interface (GUI) of the immunization management application according to an example of the instant disclosure.

FIG. 9 shows an example of a system for implementing certain aspects of the present technology.

### DETAILED DESCRIPTION

The present invention is more fully described below with reference to the accompanying figures. The following description is exemplary in that several embodiments are described (e.g., by use of the terms “preferably,” “for example,” or “in one embodiment”); however, such should not be viewed as limiting or as setting forth the only embodiments of the present invention, as the invention encompasses other embodiments not specifically recited in this description, including alternatives, modifications, and equivalents within the spirit and scope of the invention. Further, the use of the terms “invention,” “present invention,” “embodiment,” and similar terms throughout the description are used broadly and not intended to mean that the invention requires, or is limited to, any particular aspect being described or that such description is the only manner in which the invention may be made or used. Additionally, the invention may be described in the context of specific applications; however, the invention may be used in a variety of applications not specifically described.

The embodiment(s) described, and references in the specification to “one embodiment”, “an embodiment”, “an example embodiment”, etc., indicate that the embodiment(s) described may include a particular feature, structure, or characteristic. Such phrases are not necessarily referring to the same embodiment. When a particular feature, structure, or characteristic is described in connection with an embodiment, persons skilled in the art may effect such feature, structure, or characteristic in connection with other embodiments whether or not explicitly described.

In the several figures, like reference numerals may be used for like elements having like functions even in different drawings. The embodiments described, and their detailed construction and elements, are merely provided to assist in a comprehensive understanding of the invention. Thus, it is apparent that the present invention can be carried out in a variety of ways, and does not require any of the specific features described herein. Also, well-known functions or constructions are not described in detail since they would obscure the invention with unnecessary detail. Any signal arrows in the drawings/figures should be considered only as exemplary, and not limiting, unless otherwise specifically noted. Further, the description is not to be taken in a limiting sense, but is made merely for the purpose of illustrating the general principles of the invention, since the scope of the invention is best defined by the appended claims.

It will be understood that, although the terms “first,” “second,” etc. may be used herein to describe various elements, these elements should not be limited by these terms. These terms are only used to distinguish one element from another. Purely as a non-limiting example, a first element could be termed a second element, and, similarly, a second element could be termed a first element, without departing from the scope of example embodiments. As used herein, the term “and/or” includes any and all combinations of one or more of the associated listed items. As used herein, the singular forms “a”, “an,” and “the” are intended to include the plural forms as well, unless the context clearly indicates otherwise. It should also be noted that, in some alternative implementations, the functions and/or acts noted may occur out of the order as represented in at least one of the several figures. Purely as a non-limiting example, two figures shown in succession may in fact be executed substantially concurrently or may sometimes be executed in the reverse order, depending upon the functionality and/or acts described or depicted.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Conditional language, such as, among others, “can,” “could,” “might,” or “may,” unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without user input or prompting, whether these features, elements and/or steps are included or are to be performed in any particular embodiment.

Aspects of an immunization management system includes a client computing device such as a mobile computing device that communicates with one or more server computing devices. As an example, the client computing device may have an immunization management application that is executed by the client computing device. A user of the client computing device may provide healthcare information such as immunization information as well as other medical information including health history information for the user or another user such as a particular patient. The health history information may include medicines or prescriptions for the patient. The user may provide the immunization information that includes a history of vaccinations received by the patient. The immunization management application may review the history of vaccinations for the patient and compare the history of vaccinations with an official list or a governmental recommendation of vaccinations for a patient in a particular country such as the United States. The immunization management application may determine that there are one or more vaccinations that the patient is missing or overdue to receive. The immunization management application may schedule the one or more vaccinations for the patient by selecting a particular healthcare provider that may be located in a particular location associated with the patient. Additionally, the healthcare provider may be selected based on other reasons or factors such as insurance accepted by the healthcare provider. The immunization management system also may book an appointment for the patient and can schedule transportation for the patient such as transportation provided by public transportation or associated with a ride share system or service. Upon arrival at the appointment for the one or more immunizations, the immunization management application may notify one or more healthcare providers and family members or caregivers associated with the particular patient may be notified. The immunization management system may determine whether there are one or more conflicts associated with the one or more immunizations to be given at the appointment that may be based on the health history information. If there are conflicts, the healthcare providers and the family members may be notified in realtime so that adjustments can be made including rescheduling the one or more immunizations or other types of readjustments. Additionally, the immunization management system can assist patients, caregivers, or family members that may not speak English or may have difficulty speaking English.

In one example, the system may include a computing device comprising a memory storing computer-readable instructions and at least one processor to execute the instructions to receive medical information associated with a particular patient, the medical information comprising immunization records and current medicines prescribed for the particular patient, compare the immunization records for the particular patient with a recommended immunization schedule for the particular patient and determine that there is at least one immunization to be scheduled for the particular patient, schedule the at least one immunization for the particular patient based on at least one factor comprising a spoken language of the particular patient, a location of the particular patient, and acceptance of insurance for the particular patient with a healthcare provider, and generate an alert in realtime for the healthcare provider when the particular patient arrives at the healthcare provider for the at least one immunization scheduled for the particular patient by determining a location of the computing device and sending the alert to the healthcare provider, the alert indicating at least one conflict for the at least one immunization based on the medical information.

As an example, the immunization management system may have an automatic adjusting interface. When a user signs into the immunization management system, the immunization management application may select a country such as the United States and immunization and medication names may be associated with the country. As an example, TYLENOL or acetaminophen is known as paracetamol or Panadol in other locations and countries. Additionally, the immunization management system may provide one interface for a user or patient that is under eighteen years of age and another interface for a user or patient that is over eighteen years of age. In the United States, a user under the age of eighteen may be provided with a user interface that includes immunizations based on the Center for Disease Control and Prevention (CDC) standard chart. However, there is not a standard set of immunizations for users over the age of eighteen. A user that is over the age of eighteen and initially providing information to the immunization management system may be asked to enter immunization information to the interface for a user under the age of eighteen and then also enter immunization information to the interface for the user that is over the age of eighteen.

The immunization management system may provide recommendations of missing vaccinations as well as alerts if vaccinations have been received that do not appear to be appropriate or necessary. As an example, if the user is fifty years old, the immunization management system may provide a recommendation that the user have a shingles vaccine. In another example, if the user is under fifty years old and indicates that they have received the shingles vaccine, the immunization management system may send a message or an alert for a healthcare provider for more information as to why the vaccine was received before the age of fifty.

As another example, the immunization management system may allow users to share immunization and medical/dental records with a group of shared users such as family members or caregivers, among others. The immunization management system may allow a user to share a record or a representation of a record from one user to another user such as a family member associated with the sharing user. In one example, a user can share the immunization and medical/dental records and a previous user may no longer have access. For example, a foster child may move from one parent or caregiver to another parent or caregiver. Records and associated access could be transferred to the new parent and the previous parent may no longer have access.

In one example, the immunization management system can analyze data and information from one or more family members and may be able to provide predictions or recommendations such as a risk of obesity, heart attack, and cancers, among others. The immunization management system may gather medical history information from one or more related family members and may be able to provide recommendations such as particular immunizations or medications to address particular diseases. Using artificial intelligence, the immunization management system can determine diseases that may be based on genetics and provide a notification or an alert. As an example, if a parent had heart or cancer issues, children may be susceptible. In one example, the immunization management system may provide a family interface that may display one or more related family members and associated information. The immunization management system may integrate with governmental health information and alert systems to provide alerts to the users. The system may provide recommendations based on locations to seek immunization or medications.

As another example, the immunization management system may obtain information and input from a user of the immunization management application to provide alert information. In one example, the user may obtain a photograph or an image using a mobile computing device. The image may be an image of immunization records or medicine such as a receipt or a prescription bottle that may have text, a barcode, or another two-dimensional code. The immunization management application may perform optical character recognition to obtain information associated with the prescription. The immunization management application may review the prescription information, store the prescription information, and can provide future recommendations and alerts. Additionally, the user may send information associated with the prescription by emailing the image or sending a text message of the image to the immunization management system. The immunization management application may receive the image, perform optical character recognition, and review the prescription information and store the prescription information. Alternatively, the user may record information about their medical history, prescription history, or immunization history using the immunization management application by speaking to the mobile computing device. Input information may be captured by a microphone of the mobile computing device. In another example, the user may capture a QR (Quick Response) code associated with the user and their immunization or health history and the immunization management system may obtain information associated with the user and store the information associated with the user. Alternatively, the user may input prescription information or immunization information manually into an interface. The immunization management system may receive the information and store the information. The immunization management system can alert a provider about the received information and can also allow the provider or the immunization management system to alert the user if any ingredient of a prescription or medicine could adversely affect an immunization or vice versa. Additionally, the immunization management system may determine and track manufacturer doses of one or more immunizations and prescriptions and can send alerts and reminders to the user.

As another example, the immunization management system can store and provide information to a healthcare provider. In one example, if a user had surgery such as surgery on a particular limb and it cannot be used for immunization, e.g., a right arm, an alert may be sent to the healthcare provider. As another example, the user may had other medical procedures that prevent use of the limb. In one example, it may be used to obtain blood pressure or it may be connected to one or more intravenous lines. The alert may be an automated email, text, phone call, or another type of notification. This can be very important if the user or patient is unable to speak or forgets, or does not speak a same language as the healthcare provider.

As an example, the immunization management system may determine a particular location of the mobile computing device and may determine that the user is located at the healthcare provider and may determine that there is an appointment based on calendar integration information. As another example, the immunization management system may determine a particular allergy for the user such as penicillin and an alert may be displayed or sent to the healthcare provider. When the user visits the healthcare provider or an office of the healthcare provider, the immunization management application may display an interface that includes information associated with the user or patient such as a name, a date of birth, a photograph, and other information such that the user does not have to speak to provide the information. This may be particularly important if the user does not speak the same language as the healthcare provider or the patient is young or elderly.

As another example, the immunization management system may allow a non-English speaker to utilize the system. The user may be able to make one or more appointments at an English-speaking facility and obtain one or more immunizations even without speaking English. In one example, the immunization management application may translate information associated with the user or patient from English and display the information for the user in another language. Thus, the user is able to understand and may create an appointment to receive a vaccine or immunization. Additionally, the immunization management system may provide one or more GUI elements that may allow the user to book a ride or trip to a healthcare provider for the vaccine or immunization. In one example, the ride may be associated with public transportation or a ride-shared system such as a bus, taxi, train, subway, streetcar, UBER, or LYFT, among others. The immunization management system may select a particular healthcare provider and location based on a number of factors such as language spoken, proximity or distance from the user, insurance information, and access to public transportation, among others. In the event that the user does not attend the appointment, family members and/or healthcare providers may be notified.

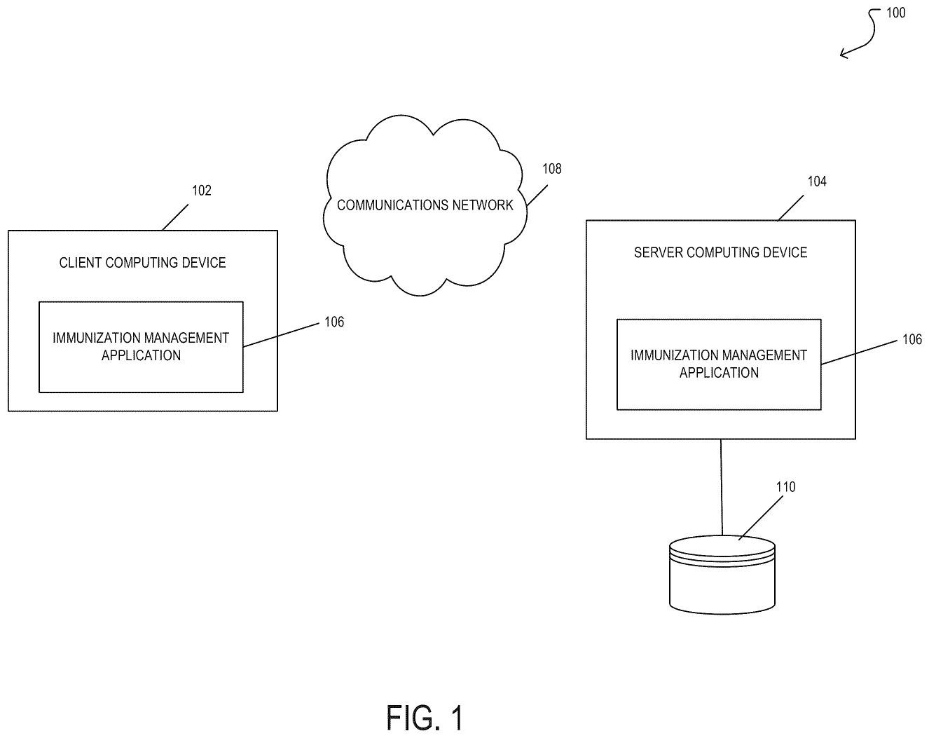

FIG. 1 is a block diagram of an immunization management system 100 according to an example of the instant disclosure. As shown in FIG. 1, the system 100 may include at least one client computing device 102 as well as at least one server computing device 104. The client computing device 102 and the server computing device 104 may have an immunization management application 106. There may be a server-side component of the immunization management application 106 as well as a client-side component of the immunization management application 106.

The client computing device 102 and the server computing device 104 may have the immunization management application 106 that may be a component of an application and/or service executable by the at least one client computing device 102 and/or the server computing device 104. For example, the immunization management application 106 may be a single unit of deployable executable code or a plurality of units of deployable executable code. According to one aspect, the immunization management application 106 may include one or more components that may be a web application, a native application, and/or a mobile application (e.g., an app) downloaded from a digital distribution application platform that allows users to browse and download applications developed with mobile software development kits (SDKs) including the App Store and GOOGLE PLAY®, among others.

The immunization management system 100 also may include a relational database management system (RDBMS) or another type of database management system such as a NoSQL database system that stores and communicates data from at least one database. The data stored in the at least one database may be associated with one or more users associated with the system as well as one or more patients associated with the system. The data stored in the database may include immunization and medicine information for each user or patient. As an example, each user of the system may have an immunization record that provides information associated with immunization records for the user before the age of eighteen and after the age of eighteen. Each user may have username/password information for use with the system.

The at least one client computing device 102 and the at least one server computing device 104 may be configured to receive data from and/or transmit data through a communication network 108. Although the client computing device 102 and the server computing device 104 are shown as a single computing device, it is contemplated each computing device may include multiple computing devices. As an example, the server computing device 104 may include one or more cloud computing devices such as virtual cloud computing servers or cloud computing instances or virtual machines that together provide the server computing device 104.

The communication network 108 can be the Internet, an intranet, or another wired or wireless communication network. For example, the communication network 106 may include a Mobile Communications (GSM) network, a code division multiple access (CDMA) network, 3^{rd} Generation Partnership Project (GPP) network, an Internet Protocol (IP) network, a wireless application protocol (WAP) network, a Wi-Fi network, a Bluetooth network, a Bluetooth Low Energy (BLE) network, an Infrared Technology (IR) network, a Radio Frequency (RF) network, an IEEE 802.15.4 Communications protocol network (e.g., Zigbee), a RS-232 Serial communication standard network, a WiMax network, a satellite communications network, or an IEEE 802.11 standards network, as well as various communications networks thereof. Other conventional and/or later developed wired and wireless networks may also be used.

The client computing device 102 may include at least one processor to process data and memory to store data. The processor processes communications, builds communications, retrieves data from memory, and stores data to memory. The processor and the memory are hardware. The memory may include volatile and/or non-volatile memory, e.g., a computer-readable storage medium such as a cache, random access memory (RAM), read only memory (ROM), flash memory, or other memory to store data and/or computer-readable executable instructions. In addition, the client computing device 102 further includes at least one communications interface to transmit and receive communications, messages, and/or signals.

The client computing device 102 could be a programmable logic controller, a programmable controller, a laptop computer, a smartphone, a personal digital assistant, a tablet computer, a standard personal computer, or another processing device. The client computing device 102 may include a display, such as a computer monitor, for displaying data and/or graphical user interfaces. The client computing device 102 may also include a Global Positioning System (GPS) hardware device for determining a particular location, an input device, such as one or more cameras or imaging devices, a keyboard or a pointing device (e.g., a mouse, trackball, pen, or touch screen) to enter data into or interact with graphical and/or other types of user interfaces. In an exemplary embodiment, the display and the input device may be incorporated together as a touch screen of the smartphone or tablet computer.

The server computing device 104 may include at least one processor to process data and memory to store data. The processor processes communications, builds communications, retrieves data from memory, and stores data to memory. The processor and the memory are hardware. The memory may include volatile and/or non-volatile memory, e.g., a computer-readable storage medium such as a cache, random access memory (RAM), read only memory (ROM), flash memory, or other memory to store data and/or computer-readable executable instructions. In addition, the server computing device 104 further includes at least one communications interface to transmit and receive communications, messages, and/or signals.

As an example, the client computing device 102 and the server computing device 104 communicate data in packets, messages, or other communications using a common protocol, e.g., Hypertext Transfer Protocol (HTTP) and/or Hypertext Transfer Protocol Secure (HTTPS). The one or more computing devices may communicate based on representational state transfer (REST) and/or Simple Object Access Protocol (SOAP). As an example, a first computer (e.g., the client computing device 102) may send a request message that is a REST and/or a SOAP request formatted using Javascript Object Notation (JSON) and/or Extensible Markup Language (XML). In response to the request message, a second computer (e.g., the server computing device 104) may transmit a REST and/or SOAP response formatted using JSON and/or XML.

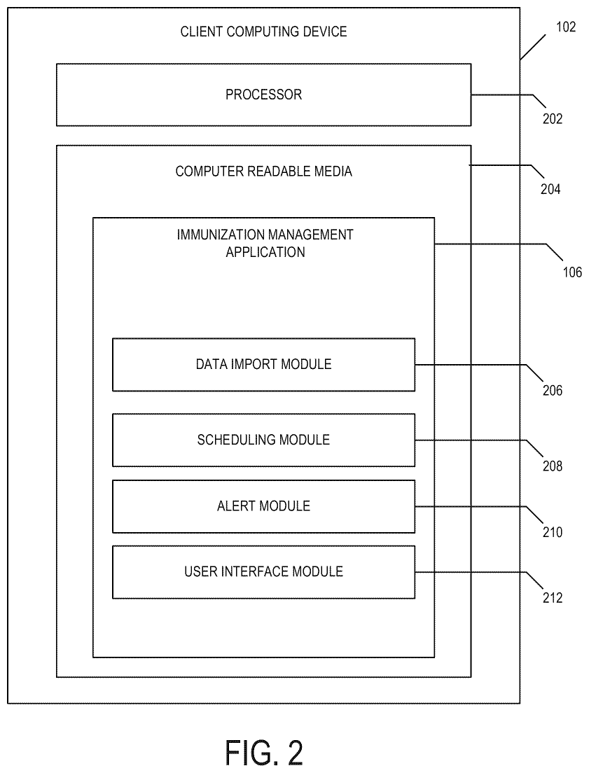

FIG. 2 illustrates a block diagram of the client computing device 102 according to an example embodiment. The client computing device 102 includes computer readable media (CRM) 204 in memory on which the immunization management application 106 or other user interface or application is stored. The computer readable media 204 may include volatile media, nonvolatile media, removable media, non-removable media, and/or another available medium that can be accessed by the processor 202. By way of example and not limitation, the computer readable media 204 comprises computer storage media and communication media. Computer storage media includes non-transitory storage memory, volatile media, nonvolatile media, removable media, and/or non-removable media implemented in a method or technology for storage of information, such as computer/machine-readable/executable instructions, data structures, program modules, or other data. Communication media may embody computer/machine-readable/executable instructions, data structures, program modules, or other data and include an information delivery media or system, both of which are hardware.

The immunization management application 106 may include a data import module 206 for obtaining data and information associated with a particular patient. As an example, a user or patient may provide information that may be imported by the immunization management application 106. The user may obtain one or more photographs or one or more images using the client computing device 102. The image may be an image of immunization records or medicine such as a receipt or a prescription bottle that may have text or a two-dimensional bar code. The data import module 206 may perform optical character recognition to obtain information associated with the prescription. The immunization management application 106 may review the prescription information, store the prescription information in the database 110, and can provide future recommendations and alerts. Additionally, the user may send information associated with the prescription by emailing the image or sending a text message of the image to the immunization management system 106. The data import module 206 may receive the image, perform optical character recognition, and review the prescription information and store the prescription information in the database 110. Alternatively, the user may record information about their medical history, prescription history, or immunization history using the immunization management application 106 by speaking to the client computing device 102. Input information may be captured by a microphone of the mobile computing device 102. In another example, the user may capture a QR (Quick Response) code associated with the user and their immunization or health history and the immunization management system may obtain information associated with the user and store the information associated with the user. Alternatively, the user may input prescription information or immunization information manually into an interface. The data import module 206 may receive the information and store the information in the database 110.

The immunization management application 106 may include a scheduling module 208 for scheduling one or more immunizations for a particular patient. As an example, the scheduling module 208 may receive medical information associated with a particular patient. The medical information can include immunization records and current (or past) medicines prescribed for the particular patient. As an example, the immunization records may be a first immunization record for when the patient is eighteen years and younger and a second immunization record or when the patient is over eighteen years of age. The scheduling module 208 may compare the immunization records for the particular patient with a recommended immunization schedule for the particular patient and determine that there is at least one immunization to be scheduled for the particular patient. In one example, the recommend immunization schedule may be provided by a government, a healthcare provider, or another entity. Based on the comparison, the scheduling module 208 may determine that there is a vaccination that was missed and is to be scheduled.

As another example, the scheduling module 208 may determine that a patient is scheduled to visit a particular country based on calendar information associated with one or more calendar applications that may communicate with the immunization management application 106 or based on one or more emails that may indicate that an upcoming trip has been booked to a particular country, e.g., Kenya. There may be a flight booked and it may be indicated in the calendar application. The scheduling module 208 may determine that there are one or more immunizations to be scheduled based on the upcoming trip to the particular country and the scheduling module 208 may schedule the one or more immunizations such that they can be completed before the trip.

The scheduling module 208 may schedule the at least one immunization for the particular patient based on at least one factor comprising a spoken language of the particular patient, a location of the particular patient, and acceptance of insurance for the particular patient with a healthcare provider, among other factors. In one example, a healthcare provider may be selected because they speak a same language as the patient, they are within ten miles of the patient, and they accept an insurance of the patient. In one example, the scheduling module 208 may schedule an appointment with a healthcare provider and may arrange transportation for the particular patient. The transportation may be public transportation or may be associated with a ride sharing service, among others.

The immunization management application 106 may include an alert module 210 for sending one or more alerts associated with one or more immunizations for a particular patient.

As an example, the alert module 210 may generate an alert in realtime for the healthcare provider when the particular patient arrives at the healthcare provider for the at least one immunization scheduled for the particular patient by determining a location of the client computing device 102 and send the alert to the healthcare provider, the alert indicating at least one conflict for the at least one immunization based on the medical information. Additionally, the alert module 210 may generate an alert in realtime for at least one family member associated with the particular patient when the particular patient arrives at the healthcare provider for the at least one immunization.

In some examples, the alert module 210 may determine that the conflict for the at least one immunization comprises a conflict with the current medicines. There may be a potential side effect associated with one of the current medicines or even a medicine that was taken in a previous time period. In another example, the alert module 210 may determine that the conflict for the at least one immunization comprises a conflict with a limb of the patient. As an example, the patient may have recently had surgery on a right arm and this arm may not be used to receive the scheduled vaccine. As another example, the user may had other medical procedures that prevent use of the limb. In one example, it may be used to obtain blood pressure or it may be connected to one or more intravenous lines. The healthcare provider may be notified and the at least one immunization may be given to another part of the patient's body, e.g., the left arm.

In addition, the immunization management application 106 includes a user interface module 212 for displaying a user interface on the display. As an example, the user interface module 212 generates a native and/or web-based graphical user interface (GUI) that accepts input and provides output viewed by users of the client computing device 102 or another computing device. The client computing device 102 or another computing device may provide realtime automatically and dynamically refreshed information. The user interface module 212 may send data to other modules of the immunization management application 106 of the client computing device 102 and retrieve data from other modules of the immunization management application 106 of the client computing device 102 asynchronously without interfering with the display and behavior of the user interface displayed by the client computing device 102 or another computing device.

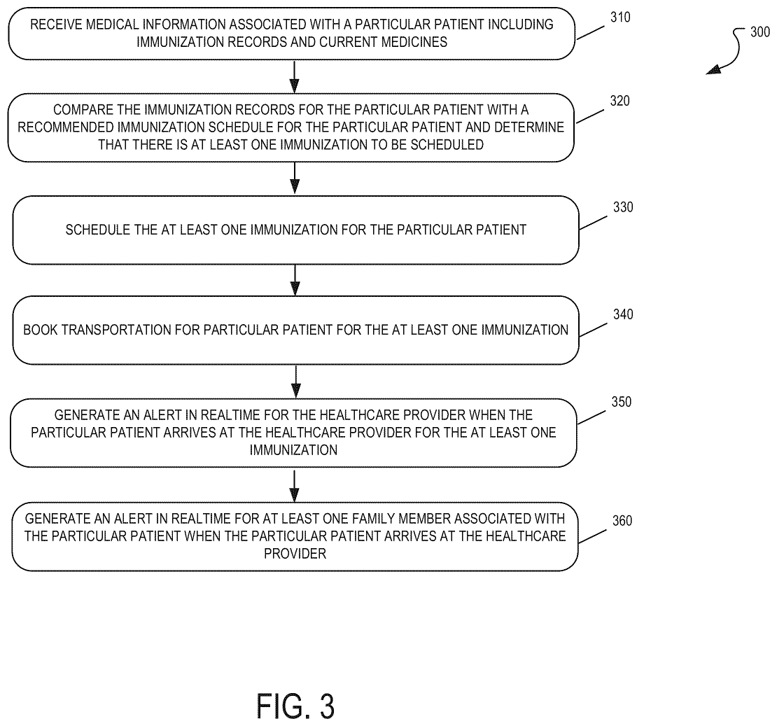

FIG. 3 illustrates an example method 300 of scheduling at least one immunization for a particular patient according to an example of the instant disclosure. Although the example method 300 depicts a particular sequence of operations, the sequence may be altered without departing from the scope of the present disclosure. For example, some of the operations depicted may be performed in parallel or in a different sequence that does not materially affect the function of the method 300. In other examples, different components of an example device or system that implements the method 300 may perform functions at substantially the same time or in a specific sequence.

According to some examples, the method 300 may include the immunization management application 106 of the client computing device 102 receiving medical information associated with a particular patient, the medical information comprising immunization records and current medicines prescribed for the particular patient at block 310.

Next, according to some examples, the method 300 may include comparing the immunization records for the particular patient with a recommended immunization schedule for the particular patient and determining that there is at least one immunization to be scheduled for the particular patient at block 320. As an example, the at least one immunization may be on the recommended immunization schedule and may have been missed.

Next, according to some examples, the method 300 may include scheduling the at least one immunization for the particular patient based on at least one factor comprising a spoken language of the particular patient, a location of the particular patient, and acceptance of insurance for the particular patient with a healthcare provider at block 330.

Next, according to some examples, the method 300 may include scheduling and booking transportation for the particular patient for the at least one immunization at block 340.

Next, according to some examples, the method 300 may include generating an alert in realtime for the healthcare provider when the particular patient arrives at the healthcare provider for the at least one immunization scheduled for the particular patient by determining a location of the computing device, and sending the alert to the healthcare provider, the alert indicating at least one conflict for the at least one immunization based on the medical information at block 350.

Next, according to some examples, the method 300 may include generating an alert in realtime for at least one family member associated with the particular patient when the particular patient arrives at the healthcare provider for the at least one immunization at block 360.

In some examples, the method 300 may include determining that the conflict for the at least one immunization comprises a conflict with the current medicines.

In some examples, the method 300 may include determining that the conflict for the at least one immunization comprises a conflict with a limb of the patient.

In some examples, the method 300 may include automatically scheduling an appointment for the at least one immunization for the particular patient by booking transportation for the particular patient using at least one of public transportation and a ride share system to transport the particular patient to the appointment.

In some examples, the method 300 may include capturing the medical information by an imaging device of the client computing device 102 that captures at least one image and determine the immunization records and the current medicines using optical character recognition (OCR) to capture text in the at least one image.

In some examples, the method 300 may include translating the immunization records and the current medicines from a first language to a second language.

In some examples, the method 300 may include receiving a request to transfer the medical information to at least one family member associated with the particular patient and sending the alert in realtime to a computing device associated with the at least one family member. In one example, a user can share the immunization and medical/dental records and a previous user may no longer have access. For example, a foster child may move from one parent or caregiver to another parent or caregiver. Records and associated access could be transferred to the new parent and the previous parent may no longer have access. In some examples, the method 300 may include receiving a request to transfer the medical information from a first user to a second user associated with the particular patient and after transferring the medical information, ending access for the first user.

In some examples, the method 300 may include sending the alert in realtime to a computing device associated with the healthcare provider.

In some examples, the method 300 may include determining the location of the computing device using at least one of a global positioning (GPS) hardware of the client computing device 102, cellular triangulation, Wi-Fi positioning, and Bluetooth positioning.

In some examples, the method 300 may include storing the medical information for the particular patient in the database 110 and determining at least one family member associated with the particular patient.

In some examples, the method 300 may include determining at least one potential future medical issue for the at least one family member based on the medical information for the particular patient. The at least one potential medical issues may be associated with hereditary traits and family history information.

In some examples, the method 300 may include determining the at least one potential medical issue using artificial intelligence.

In some examples, the method 300 may include determining the at least one conflict based on a determined side effect using artificial intelligence.

As an example, a user can provide information such as a medication they are taking or an immunization they have received. The system 100 can determine ingredients and how long they are active by performing one or more queries in various health databases that may have that information such as databases provided by John Hopkins and Mayo Clinic, among others. Based on that information, the method 300 may include providing an alert to a user about not taking the medicine or indicating that it is OK to take the medicine. If two or more medicines cannot be mixed, then a notification can be provided. If the medicine can be mixed but after a particular period of time, then an appropriate notification is sent. If a user is taking two or more medications that do the same thing, then a notification can be sent out because it may be harmful to have a dose that is too high.

A number of medicines may conflict with one another but each medicine is made up of many ingredients and that conflict can be difficult to determine. Even when the medicines are conflicting, the information can be hidden or unknown if a user is going to two different doctors and they prescribe the medications without the ability to know what the user is taking. Often, the systems used by the healthcare providers do not communicate such information between the systems. Many times, prescription or over-the-counter (OTC) medications can conflict with vitamins, minerals, or other dietary supplements. Many times, a religious person may not want medicines because many times they are made of animals. In these cases, the method 300 may determine that there are one or more conflicts and provide one or more alerts.

In some examples, the method 300 may include displaying a graphical user interface (GUI) that displays a first immunization interface when the particular patient is eighteen years of age or less and that displays a second immunization interface when the particular patient is above eighteen years of age.

In some examples, the method 300 may include displaying a GUI that comprises a timeline of scheduled immunizations for the particular patient and indicates whether the particular patient has received each of the scheduled immunizations. The scheduled immunizations can be shown in a first color when they have been received, in a second color when they are overdue, and in a third color when they are upcoming. The GUI can be based on a country of the particular patient and the immunization records and the current medicines prescribed for the particular patient are displayed as known in the country of the particular patient. The immunization schedule may be associated with a particular country and may be different from one country to another.

In some examples, the method 300 may include receiving the medical information comprising a recording of speech that can be captured by the client computing device 102 and performing natural language processing on the recording of the speech to determine the medical information.

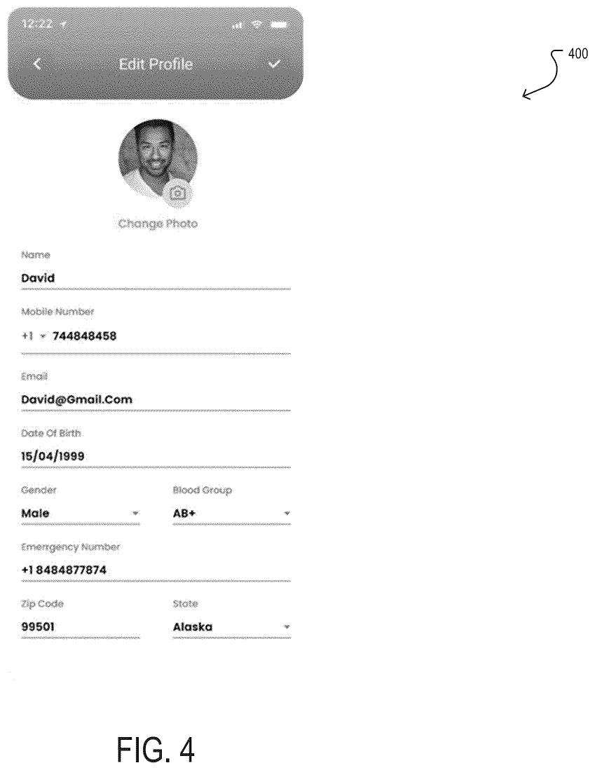

FIGS. 4-8 show example screenshots of a graphical user interface (GUI) of the immunization management application 106 according to an example of the instant disclosure.

As shown in FIG. 4, there is an example screenshot 400 of an example GUI that shows a profile of a user or patient having an account or information associated with the immunization management application 106. As shown in FIG. 4, the system 100 may store information including one or more images of the patient, a name of the patient, one or more phone numbers associated with the patient, one or more email addresses associated with the patient, a date of birth of the patient, a gender of the patient, a blood type or blood group of the patient, one or more emergency contact telephone numbers or emergency contact information for the patient, a zip code for the patient, and a state for the patient, among other information. As an example, the user or patient could show the GUI to a healthcare provider when being seen rather than provide any of the information verbally to further protect the privacy of the information. The patient also may be unable to communicate for various reasons such as an inability to speak a same language as the healthcare provider.

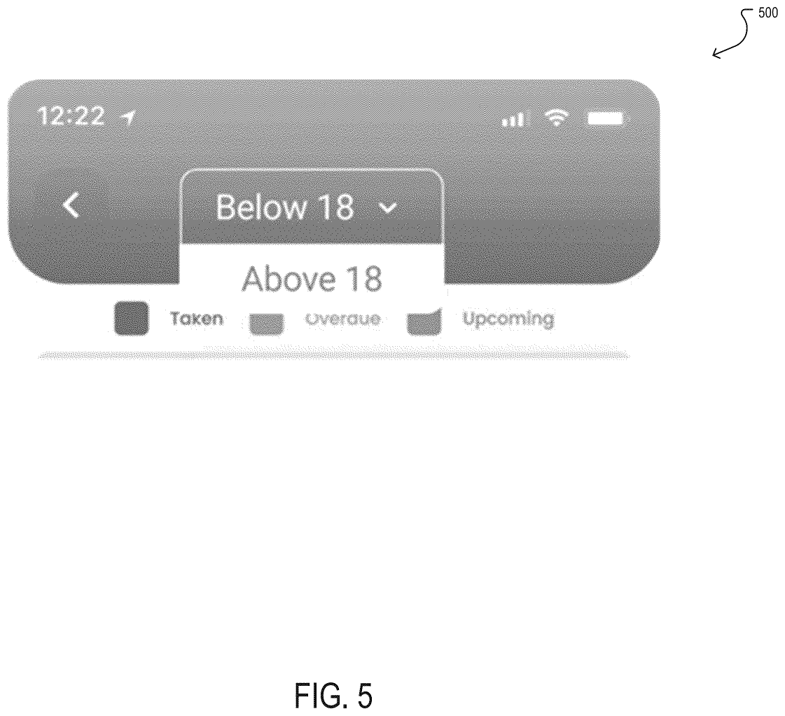

As shown in FIG. 5, there is an example screenshot 500 of an example GUI that shows that the user can display a first immunization interface associated with scheduled immunizations that are to occur at age eighteen and less and a second immunization interface with scheduled immunizations that are to occur after age eighteen. The user can view the first immunization interface by selecting a GUI element associated with the first immunization interface and can view the second immunization interface by selecting another GUI element associated with the second immunization interface.

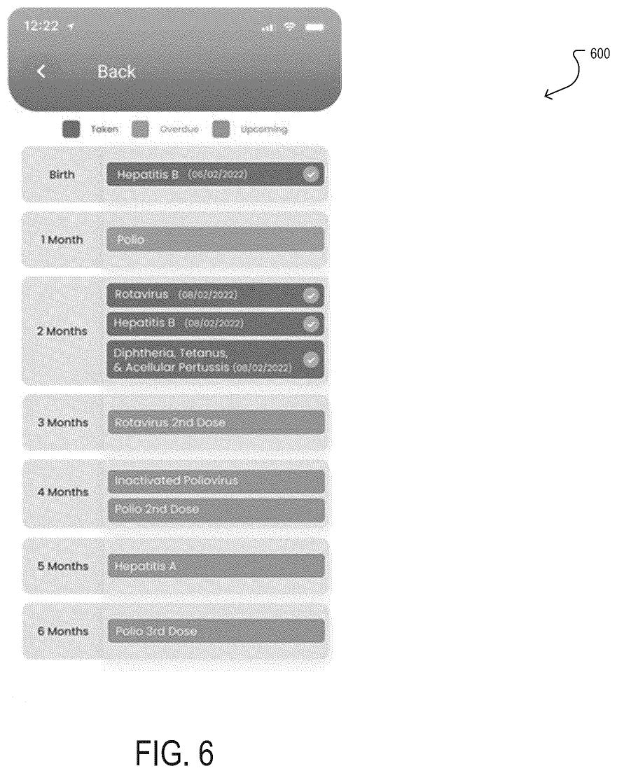

As shown in FIG. 6, there is an example screenshot 600 of an example GUI that shows the first immunization interface for a particular patient. As shown in FIG. 6, there are scheduled immunizations for a particular patient including immunizations scheduled for birth (Hepatitis B), one month (Polio), two months (Rotavirus, Hepatitis B, Diphtheria, Tetanus, & Acellular Pertussis), three months (Rotavirus 2^{nd} Dose), four months (Inactivated Poliovirus, Polio 2^{nd} Dose), five months (Hepatitis A), six months (Polio 3^{rd} Dose), and so on. Immunizations that have been received or taken can be shown in first color such as green and there may be an indication that they have been received on a particular date along with a checkmark or another type of indication. Immunizations that are overdue may be shown in a second color such as red. Immunizations that are upcoming may be shown in a third color such as blue.

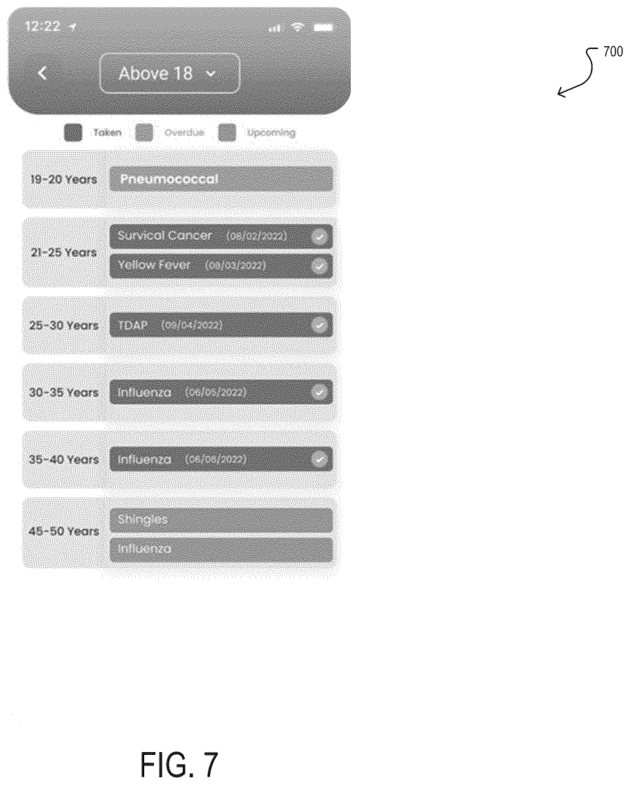

As shown in FIG. 7, there is an example screenshot 700 of an example GUI that shows the second immunization interface for a particular patient. As shown in FIG. 7, there are scheduled immunizations for a particular patient including immunizations to occur at nineteen to twenty years of age (Pneumococcal), immunizations that are to occur from twenty-one years of age to twenty-five years of age (Cervical Cancer, Yellow Fever), immunizations that are to occur from twenty-five years of age to thirty years of age (TDAP), immunizations that are to occur from thirty years of age to thirty-five years of age (Influenza), immunizations that are to occur from thirty-five years of age to forty years of age (Influenza), and immunizations that are to occur from forty-five years of age to fifty years of age (Shingles, Influenza), and so on. Again, immunizations that have been received or taken can be shown in a first color such as green and there may be an indication that they have been received on a particular date along with a checkmark or another type of indication. Immunizations that are overdue may be shown in a second color such as red. Immunizations that are upcoming may be shown in a third color such as blue.

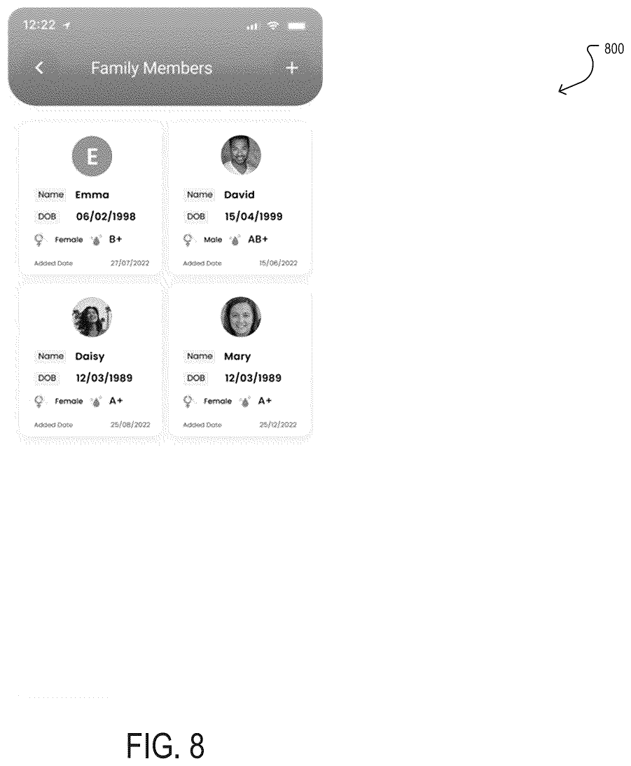

As shown in FIG. 8, there is an example screenshot 800 of an example GUI that shows one or more family members associated with a particular patient. As shown in FIG. 8, there are four family members including Emma, David, Daisy, and Mary. A particular patient may be asked to accept and allow each family member to view the medical information (e.g., the immunization record) associated with the particular patient.

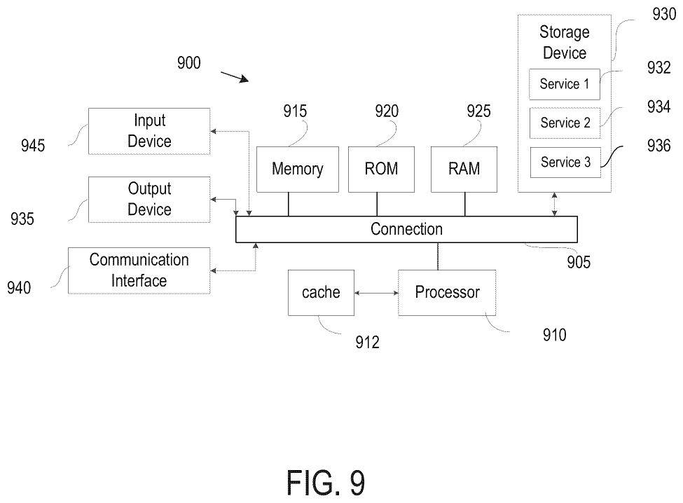

FIG. 9 shows an example of computing system 900, which can be for example any computing device making up the computing device such as the client computing device 102, the server computing device 104, or any component thereof in which the components of the system are in communication with each other using connection 905. Connection 905 can be a physical connection via a bus, or a direct connection into processor 910, such as in a chipset architecture. Connection 905 can also be a virtual connection, networked connection, or logical connection.

In some embodiments, computing system 900 is a distributed system in which the functions described in this disclosure can be distributed within a datacenter, multiple data centers, a peer network, etc. In some embodiments, one or more of the described system components represents many such components each performing some or all of the function for which the component is described. In some embodiments, the components can be physical or virtual devices.

Example system 900 includes at least one processing unit (CPU or processor) 910 and connection 905 that couples various system components including system memory 915, such as read-only memory (ROM) 920 and random access memory (RAM) 925 to processor 910. Computing system 900 can include a cache of high-speed memory 912 connected directly with, in close proximity to, or integrated as part of processor 910.

Processor 910 can include any general purpose processor and a hardware service or software service, such as services 932, 934, and 936 stored in storage device 930, configured to control processor 910 as well as a special-purpose processor where software instructions are incorporated into the actual processor design. Processor 910 may essentially be a completely self-contained computing system, containing multiple cores or processors, a bus, memory controller, cache, etc. A multi-core processor may be symmetric or asymmetric.

To enable user interaction, computing system 900 includes an input device 945, which can represent any number of input mechanisms, such as a microphone for speech, a touch-sensitive screen for gesture or graphical input, keyboard, mouse, motion input, speech, etc. Computing system 900 can also include output device 935, which can be one or more of a number of output mechanisms known to those of skill in the art. In some instances, multimodal systems can enable a user to provide multiple types of input/output to communicate with computing system 900. Computing system 900 can include communications interface 940, which can generally govern and manage the user input and system output. There is no restriction on operating on any particular hardware arrangement, and therefore the basic features here may easily be substituted for improved hardware or firmware arrangements as they are developed.

Storage device 930 can be a non-volatile memory device and can be a hard disk or other types of computer readable media which can store data that are accessible by a computer, such as magnetic cassettes, flash memory cards, solid state memory devices, digital versatile disks, cartridges, random access memories (RAMs), read-only memory (ROM), and/or some combination of these devices.

The storage device 930 can include software services, servers, services, etc., that when the code that defines such software is executed by the processor 910, it causes the system to perform a function. In some embodiments, a hardware service that performs a particular function can include the software component stored in a computer-readable medium in connection with the necessary hardware components, such as processor 910, connection 905, output device 935, etc., to carry out the function.

For clarity of explanation, in some instances, the present technology may be presented as including individual functional blocks including functional blocks comprising devices, device components, steps or routines in a method embodied in software, or combinations of hardware and software.

Any of the steps, operations, functions, or processes described herein may be performed or implemented by a combination of hardware and software services or services, alone or in combination with other devices. In some embodiments, a service can be software that resides in memory of a client device and/or one or more servers of a content management system and perform one or more functions when a processor executes the software associated with the service. In some embodiments, a service is a program or a collection of programs that carry out a specific function. In some embodiments, a service can be considered a server. The memory can be a non-transitory computer-readable medium.

In some embodiments, the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Methods according to the above-described examples can be implemented using computer-executable instructions that are stored or otherwise available from computer-readable media. Such instructions can comprise, for example, instructions and data which cause or otherwise configure a general purpose computer, special purpose computer, or special purpose processing device to perform a certain function or group of functions. Portions of computer resources used can be accessible over a network. The executable computer instructions may be, for example, binaries, intermediate format instructions such as assembly language, firmware, or source code. Examples of computer-readable media that may be used to store instructions, information used, and/or information created during methods according to described examples include magnetic or optical disks, solid-state memory devices, flash memory, USB devices provided with non-volatile memory, networked storage devices, and so on.

Devices implementing methods according to these disclosures can comprise hardware, firmware and/or software, and can take any of a variety of form factors. Typical examples of such form factors include servers, laptops, tablets, smartphones, small form factor personal computers, personal digital assistants, and so on. The functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are means for providing the functions described in these disclosures.

Illustrative examples of the disclosure include:

Aspect 1: A system comprising: a computing device comprising a memory storing computer-readable instructions and at least one processor to execute the instructions to receive medical information associated with a particular patient, the medical information comprising immunization records and current medicines prescribed for the particular patient, compare the immunization records for the particular patient with a recommended immunization schedule for the particular patient and determine that there is at least one immunization to be scheduled for the particular patient, schedule the at least one immunization for the particular patient based on at least one factor comprising a spoken language of the particular patient, a location of the particular patient, and acceptance of insurance for the particular patient with a healthcare provider, and generate an alert in realtime for the healthcare provider when the particular patient arrives at the healthcare provider for the at least one immunization scheduled for the particular patient by determining a location of the computing device and sending the alert to the healthcare provider, the alert indicating at least one conflict for the at least one immunization based on the medical information.

Aspect 2: The system of Aspect 1, the at least one processor further to determine that the conflict for the at least one immunization comprises a conflict with the current medicines.

Aspect 3: The system of any of Aspects 1 and 2, the at least one processor further to determine that the conflict for the at least one immunization comprises a conflict with a limb of the patient.

Aspect 4: The system of any of Aspects 1 to 3, the at least one processor further to automatically schedule an appointment for the at least one immunization for the particular patient by booking transportation for the particular patient using at least one of public transportation and a ride share system to transport the particular patient to the appointment.

Aspect 5: The system of any of Aspects 1 to 4, the at least one processor further to capture the medical information by an imaging device of the computing device that captures at least one image and determine the immunization records and the current medicines using optical character recognition (OCR) to capture text in the at least one image.

Aspect 6: The system of any of Aspects 1 to 5, the at least one processor further translate the immunization records and the current medicines from a first language to a second language.

Aspect 7: The system of any of Aspects 1 to 6, the at least one processor further to receive a request to transfer the medical information to at least one family member associated with the particular patient and send the alert in realtime to a computing device associated with the at least one family member.

Aspect 8: The system of any of Aspects 1 to 7, the at least one processor further to receive a request to transfer the medical information from a first user to a second user associated with the particular patient and after transferring the medical information, ending access for the first user.

Aspect 9: The system of any of Aspects 1 to 8, the at least one processor further to send the alert in realtime to a computing device associated with the healthcare provider.

Aspect 10: The system of any of Aspects 1 to 9, the at least one processor further to determine the location of the computing device using at least one of a global positioning (GPS) hardware of the computing device, cellular triangulation, Wi-Fi positioning, and Bluetooth positioning.

Aspect 11: The system of any of Aspects 1 to 10, the at least one processor further to store the medical information for the particular patient in a database and determine at least one family member associated with the particular patient.

Aspect 12: The system of any of Aspects 1 to 11, the at least one processor further to determine at least one potential future medical issue for the at least one family member based on the medical information for the particular patient.

Aspect 13: The system of any of Aspects 1 to 12, the at least one processor further to determine the at least one potential medical issue using artificial intelligence.

Aspect 14: The system of any of Aspects 1 to 13, the at least one processor further to determine the at least one conflict based on a determined side effect using artificial intelligence.

Aspect 15: The system of any of Aspects 1 to 14, the at least one processor further to display a graphical user interface (GUI) that displays a first immunization interface when the particular patient is eighteen years of age or less and that displays a second immunization interface when the particular patient is above eighteen years of age.

Aspect 16: The system of any of Aspects 1 to 15, the at least one processor further to display a GUI that comprises a timeline of scheduled immunizations for the particular patient and indicates whether the particular patient has received each of the scheduled immunizations and scheduled immunizations are shown in a first color when they have been received, in a second color when they are overdue, and in a third color when they are upcoming.

Aspect 17: The system of any of Aspects 1 to 16, wherein the GUI is based on a country of the particular patient and the immunization records and the current medicines prescribed for the particular patient are displayed as known in the country of the particular patient.

Aspect 18: The system of any of Aspects 1 to 17, the at least one processor further to receive the medical information comprising a recording of speech and perform natural language processing on the recording of the speech to determine the medical information.

Aspect 19: A method comprising receiving, by at least one processor of a mobile computing device, medical information associated with a particular patient, the medical information comprising immunization records and current medicines prescribed for the particular patient, comparing, by the at least one processor of the mobile computing device, the immunization records for the particular patient with a recommended immunization schedule for the particular patient and determining that there is at least one immunization to be scheduled for the particular patient, scheduling, by the at least one processor of the mobile computing device, the at least one immunization for the particular patient based on at least one factor comprising a spoken language of the particular patient, a location of the particular patient, and acceptance of insurance for the particular patient with a healthcare provider, and generating, by the at least one processor of the mobile computing device, an alert in realtime for the healthcare provider when the particular patient arrives at the healthcare provider for the at least one immunization scheduled for the particular patient by determining a location of the computing device and sending the alert to the healthcare provider, the alert indicating at least one conflict for the at least one immunization based on the medical information.

Aspect 20: A non-transitory computer-readable storage medium comprising instructions stored thereon that, when executed by a mobile computing device cause the mobile computing device to perform operations, the operations including receiving medical information associated with a particular patient, the medical information comprising immunization records and current medicines prescribed for the particular patient, comparing the immunization records for the particular patient with a recommended immunization schedule for the particular patient and determining that there is at least one immunization to be scheduled for the particular patient, scheduling the at least one immunization for the particular patient based on at least one factor comprising a spoken language of the particular patient, a location of the particular patient, and acceptance of insurance for the particular patient with a healthcare provider, and generating an alert in realtime for the healthcare provider when the particular patient arrives at the healthcare provider for the at least one immunization scheduled for the particular patient by determining a location of the computing device and sending the alert to the healthcare provider, the alert indicating at least one conflict for the at least one immunization based on the medical information.

What is claimed is:

1. A system comprising:
a computing device comprising a memory storing computer-readable instructions; and
at least one processor to execute the instructions to:
receive medical information associated with a particular patient, the medical information comprising immunization records and current medicines prescribed for the particular patient;
compare the immunization records for the particular patient with a recommended immunization schedule for the particular patient and determine that there is at least one immunization to be scheduled for the particular patient;
schedule the at least one immunization for the particular patient based on at least one factor comprising a spoken language of the particular patient, a location of the particular patient, and acceptance of insurance for the particular patient with a healthcare provider; and
generate an alert in realtime for the healthcare provider when the particular patient arrives at the healthcare provider for the at least one immunization scheduled for the particular patient by determining a location of the computing device and sending the alert to the healthcare provider, the alert indicating at least one conflict for the at least one immunization based on the medical information.

2. The system of claim 1, the at least one processor further to determine that the conflict for the at least one immunization comprises a conflict with the current medicines.

3. The system of claim 1, the at least one processor further to determine that the conflict for the at least one immunization comprises a conflict with a limb of the patient.

4. The system of claim 1, the at least one processor further to automatically schedule an appointment for the at least one immunization for the particular patient by booking transportation for the particular patient using at least one of public transportation and a ride share system to transport the particular patient to the appointment.

5. The system of claim 1, the at least one processor further to capture the medical information by an imaging device of the computing device that captures at least one image and determine the immunization records and the current medicines using optical character recognition (OCR) to capture text in the at least one image.

6. The system of claim 1, the at least one processor further translate the immunization records and the current medicines from a first language to a second language.

7. The system of claim 1, the at least one processor further to receive a request to transfer the medical information to at least one family member associated with the particular patient and send the alert in realtime to a computing device associated with the at least one family member.

8. The system of claim 1, the at least one processor further to receive a request to transfer the medical information from a first user to a second user associated with the particular patient and after transferring the medical information, ending access for the first user.

9. The system of claim 1, the at least one processor further to send the alert in realtime to a computing device associated with the healthcare provider.

10. The system of claim 1, the at least one processor further to determine the location of the computing device using at least one of a global positioning (GPS) hardware of the computing device, cellular triangulation, Wi-Fi positioning, and Bluetooth positioning.

11. The system of claim 1, the at least one processor further to store the medical information for the particular patient in a database and determine at least one family member associated with the particular patient.

12. The system of claim 11, the at least one processor further to determine at least one potential future medical issue for the at least one family member based on the medical information for the particular patient.

13. The system of claim 12, the at least one processor further to determine the at least one potential medical issue using artificial intelligence.

14. The system of claim 1, the at least one processor further to determine the at least one conflict based on a determined side effect using artificial intelligence.

15. The system of claim 1, the at least one processor further to display a graphical user interface (GUI) that displays a first immunization interface when the particular patient is eighteen years of age or less and that displays a second immunization interface when the particular patient is above eighteen years of age.

16. The system of claim 15, the at least one processor further to display a GUI that comprises a timeline of scheduled immunizations for the particular patient and indicates whether the particular patient has received each of the scheduled immunizations and scheduled immunizations are shown in a first color when they have been received, in a second color when they are overdue, and in a third color when they are upcoming.

17. The system of claim 15, wherein the GUI is based on a country of the particular patient and the immunization records and the current medicines prescribed for the particular patient are displayed as known in the country of the particular patient.

18. The system of claim 1, the at least one processor further to receive the medical information comprising a recording of speech and perform natural language processing on the recording of the speech to determine the medical information.

19. A method, comprising:
receiving, by at least one processor of a mobile computing device, medical information associated with a particular patient, the medical information comprising immunization records and current medicines prescribed for the particular patient;
comparing, by the at least one processor of the mobile computing device, the immunization records for the particular patient with a recommended immunization schedule for the particular patient and determining that there is at least one immunization to be scheduled for the particular patient;
scheduling, by the at least one processor of the mobile computing device, the at least one immunization for the particular patient based on at least one factor comprising a spoken language of the particular patient, a location of the particular patient, and acceptance of insurance for the particular patient with a healthcare provider; and
generating, by the at least one processor of the mobile computing device, an alert in realtime for the healthcare provider when the particular patient arrives at the healthcare provider for the at least one immunization scheduled for the particular patient by determining a location of the computing device and sending the alert to the healthcare provider, the alert indicating at least one conflict for the at least one immunization based on the medical information.

20. A non-transitory computer-readable storage medium comprising instructions stored thereon that, when executed by a mobile computing device cause the mobile computing device to perform operations, the operations comprising:
receiving medical information associated with a particular patient, the medical information comprising immunization records and current medicines prescribed for the particular patient;
comparing the immunization records for the particular patient with a recommended immunization schedule for the particular patient and determining that there is at least one immunization to be scheduled for the particular patient;
scheduling the at least one immunization for the particular patient based on at least one factor comprising a spoken language of the particular patient, a location of the particular patient, and acceptance of insurance for the particular patient with a healthcare provider; and
generating an alert in realtime for the healthcare provider when the particular patient arrives at the healthcare provider for the at least one immunization scheduled for the particular patient by determining a location of the computing device and sending the alert to the healthcare provider, the alert indicating at least one conflict for the at least one immunization based on the medical information.
